(19) United States
(12) Patent Application Publication
(10) Pub. No.: US 2024/0309376 A1
(43) Pub. Date: Sep. 19, 2024

(54) POLYNUCLEOTIDE COMPOSITIONS, RELATED FORMULATIONS, AND METHODS OF USE THEREOF

(71) Applicant: ReCode Therapeutics, Inc., Menlo Park, CA (US)

(72) Inventors: Michael Torres, Irving, TX (US); Dmitri Boudko, Irving, TX (US); Ella Meleshkevitch, Irving, TX (US); Melissa Coquelin, Dallas, TX (US)

(21) Appl. No.: 18/532,995

(22) Filed: Dec. 7, 2023

Related U.S. Application Data
(63) Continuation of application No. PCT/US2022/032643, filed on Jun. 8, 2022.
(60) Provisional application No. 63/208,957, filed on Jun. 9, 2021.

(51) Int. Cl. C12N 15/113; A61K 9/00; A61K 9/51; A61P 11/00

(52) U.S. Cl. CPC C12N 15/113; A61K 9/0078; A61K 9/5123; A61P 11/00; C12N 2310/11; C12N 2310/531

(57) ABSTRACT

Compositions of polynucleotide(s), pharmaceutical compositions thereof, and methods of use thereof are disclosed. A polynucleotide may be or encode a synthetic transfer ribonucleic acid (tRNA). The polynucleotide may be assembled with a lipid composition for delivery to a cell or an organ, such as a lung cell or a lung of a subject. Methods for enhancing an expression or activity of cystic fibrosis transmembrane conductance regulator (CFTR) protein in a cell are provided. Methods for treating a subject having or suspected of having a CFTR-associated condition are also provided.

### CROSS-REFERENCE

This application is a continuation of International Application No. PCT/US2022/032643, filed Jun. 8, 2022, which claims the benefit of U.S. Provisional Application Ser. No. 63/208,957, filed on Jun. 9, 2021, the entirety of which is hereby incorporated by reference herein.

### BACKGROUND

Nucleic acids, such as transfer RNA (tRNA) may be used by cells to express proteins and polypeptides. Some cells may be deficient in a certain protein or nucleic acid and result in disease states. A cell can also take up and use exogenous tRNA which can be used in protein synthesis reaction, but many factors influence efficient uptake of the tRNA and translation. For instance, the immune system recognizes many exogenous RNAs as foreign and triggers a response that is aimed at inactivating the RNAs.

### SUMMARY

Provided here are composition and methods for delivery of nucleic acids. Nucleic acids may be used as a therapeutic. In particular, a tRNA may be delivered to a cell of a subject. Upon delivery of a nucleic acid to a cell, the nucleic acid may be used to synthesize a polypeptide. In the case of a cell or subject with a disease or disorder, the nucleic acid may be effective at acting as a therapeutic by increasing the expression of a polypeptide. In cases, where a disorder or disease is caused or correlated to aberrant expression or activity of polypeptide, the increased in expression of the polypeptide may be beneficial. However, the cells may have limited uptake of exogenous nucleic acids and the delivery of the nucleic acids may benefit from compositions that allow for increase uptake of a nucleic acid.

Additionally, therapeutic could benefit from organ specific delivery. Many different types of compounds such as chemotherapeutic agents exhibit significant cytotoxicity. If these compounds could be better directed towards delivery to the desired organs, then fewer off target effects will be seen.

In an aspect, the present disclosure provides a composition comprising a synthetic transfer ribonucleic acid (tRNA) assembled with a lipid composition, which lipid composition comprises a zwitterionic lipid, wherein the composition is an aerosol composition.

In another aspect, the present disclosure provides composition comprising a synthetic transfer ribonucleic acid (tRNA) assembled with a lipid composition, which lipid composition comprises a zwitterionic lipid, wherein the composition is formulated for aerosol administration. In some embodiments, the composition has a droplet size from 0.5 micron (μm) to 10 μm. In some embodiments, the composition has a median droplet size from 0.5 μm to 10 μm. In some embodiments, the composition has an average droplet size from 0.5 μm to 10 μm. In some embodiments, the synthetic tRNA is a folded tRNA. In some embodiments, the folded tRNA comprises a T-arm, a D-arm, an anticodon arm, a variable loop, an acceptor stem, or a combination thereof. In some embodiments, the synthetic tRNA comprises an anticodon arm that is configured to recognize a premature stop codon. In some embodiments, the synthetic tRNA comprises an acceptor stem that is configured to be operably linked to an arginine In some embodiments, the tRNA comprises a polynucleotide sequence having at least about 800, 85%, 860, 870, 880, 89%, 90%, 91%, 92%, 93%, 94%, 95%, %%, 97%, 98%, or 99% identity to a sequence selected from SEQ ID NOs: 1-20. The composition of any one of claims 1-10, wherein a (e.g., weight or mass) ratio of the zwitterionic lipid to the synthetic tRNA is of no more than about 50:1, 40:1, 30:1, or 20:1. In some embodiments, the lipid composition comprises the zwitterionic lipid at a molar percentage of about 1% to about 60%. In some embodiments, the lipid composition further comprises a steroid or steroid derivative In some embodiments, the composition comprises the steroid or steroid derivative at a molar percentage of about 20% to about 60%. In some embodiments, the lipid composition further comprises a polymer-conjugated lipid. In some embodiments, the lipid composition comprises the polymer-conjugated lipid at a molar percentage of about 0.5% to about 12%. In some embodiments, a molar ratio of nitrogen in the lipid composition to phosphate in the synthetic tRNA (N/P ratio) is of no more than about 50:1, 40:1, 30:1, 20:1, or 10:1. In some embodiments, the zwitterionic lipid comprises a sulfonate anion. In some embodiments, the zwitterionic lipid further comprises a quaternary ammonium cation. In some embodiments, the zwitterionic lipid comprises an alkylated or alkenylated phosphate anion. In some embodiments, the alkylated or alkenylated phosphate anion has a structural formula

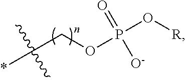

wherein R is alkyl or alkenyl; n is 1, 2, 3, 4, 5, or 6; and * indicates a point of attachment of the alkylated or alkenylated phosphate anion. In some embodiments, the * indicates a point of attachment of the alkylated or alkenylated phosphate anion to a quaternary ammonium cation.

In another aspect, the present disclosure provides a method for enhancing an expression or activity of cystic fibrosis transmembrane conductance regulator (CFTR) protein in a cell, the method comprising: contacting the cell with a composition comprising a transfer ribonucleic acid (tRNA) assembled with a lipid composition to introduce an amino acid into a growing peptide chain of a CFTR protein in the cell, thereby yielding a therapeutically effective amount or activity of a functional variant of CFTR protein in the cell at least 48 hours after contacting, optionally wherein the therapeutically effective activity of the functional variant of CFTR protein is determined by measuring a change in a transepithelial ion transport characteristic of a plurality of cells comprising the cell as compared to that of a reference plurality of cells in absence of the contacting.

In another aspect, the present disclosure provides a method for enhancing an expression or activity of cystic fibrosis transmembrane conductance regulator (CFTR) protein in a cell of a subject exhibiting or suspected of exhibiting a mutation in a CFTR gene, the method comprising: contacting the cell with a composition comprising a transfer ribonucleic acid (tRNA) assembled with a lipid composition to introduce an amino acid into a growing peptide chain of a CFTR protein in the cell at a position corresponding to the mutation in the CFTR gene of the subject, thereby yielding a therapeutically effective amount or activity of a functional variant of CFTR protein in the cell, optionally wherein the therapeutically effective activity of the functional variant of CFTR protein is determined by measuring a change in a transepithelial ion transport characteristic of a plurality of cells comprising the cell as compared to that of a reference plurality of cells in absence of the contacting. In some embodiments, the method yields a therapeutically effective amount or activity of a functional variant of CFTR protein in the cell at least 72 hours after contacting. In some embodiments, the contacting is repeated. In some embodiments, the contacting is at least once a week. In some embodiments, the contacting is at least twice a week. In some embodiments, the method yields a therapeutically effective amount or activity of a functional variant of CFTR protein in the cell at least 24 hours after each contacting. In some embodiments, the contacting is a first contacting, and wherein the method comprises a second contacting, optionally, performed at least about 1, 2, or 3 day(s) after the first contacting. In some embodiments, the methods further comprise a third contacting, optionally wherein the third contacting is performed at least about 1, 2, or 3 day(s) after the second contacting. In some embodiments, the method yields a therapeutically effective amount or activity of a functional variant of CFTR protein in the cell at least 24 hours after a second contacting. In some embodiments, the method yields a therapeutically effective amount or activity of a functional variant of CFTR protein in the cell at least 24 hours after a third contacting. In some embodiments, the composition in each contacting is identical. In some embodiments, the cell is a lung airway cell. In some embodiments, the cell is a lung secretory cell.

In some embodiments, the cell is a bronchial epithelial cell. In some embodiments, the cell is undifferentiated. In some embodiments, the cell is differentiated. In some embodiments, the cell is derived from the subject. In some embodiments, the contacting is in vivo. In some embodiments, the contacting is in vitro. In some embodiments, the contacting is ex vivo. In some embodiments, the functional variant of CFTR protein is a wild-type CFTR protein. In some embodiments, the functional variant of CFTR protein is a full-length CFTR protein. In some embodiments, the therapeutically effective activity of the functional variant of CFTR protein corresponds to a transepithelial current of at least about 2 micro-Ampere (μA), e.g., as determined in an in vitro assay. In some embodiments, the therapeutically effective activity of the functional variant of CFTR protein corresponds to a transepithelial current from about 2 micro-Ampere (μA) to about 30 μA, e.g., as determined in an in vitro assay. In some embodiments, the therapeutically effective activity of the functional variant of CFTR protein corresponds to a transepithelial current of at least about 2 micro-Ampere (μA) per squared centimeter per minute (μA·cm⁻²·min⁻¹), e.g., as determined in an in vitro assay. In some embodiments, the therapeutically effective activity of the functional variant of CFTR protein corresponds to a transepithelial current from about 2 micro-Ampere (μA) per squared centimeter per minute (μA·cm⁻²·min⁻¹) to about 30 μA·cm⁻²·min⁻¹, e.g., as determined in an in vitro assay. In some embodiments, the method increases an amount or activity of the functional variant of CFTR protein in the cell (e.g., by at least about 1.1-fold) relative to a corresponding control (e.g., that of a corresponding cell absent the contacting). In some embodiments, the method enhances (e.g., chloride) ion transport in the cell (e.g., by at least about 1.1-fold) relative to a corresponding control (e.g., that of a corresponding cell absent the contacting). In some embodiments, the mutation is a loss-of-function mutation. In some embodiments, the mutation is a nonsense or frameshift mutation. In some embodiments, the mutation is in one or more of exons 11-27 of CFTR gene. In some embodiments, the mutation is R553X. In some embodiments, the tRNA is a suppressor tRNA. In some embodiments, the tRNA comprises a polynucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 1-20. In some embodiments, the composition comprising the tRNA assembled with the lipid composition is an aerosol. In some embodiments, the composition is formulated for apical delivery. In some embodiments, the composition is formulated for nebulization.

In another aspect, the present disclosure provides a method for treating a subject having or suspected of having a cystic fibrosis transmembrane conductance regulator (CFTR)-associated condition, the method comprising administering to the subject a composition as described elsewhere herein. In some embodiments, the CFTR-associated condition is cystic fibrosis, hereditary emphysema, or chronic obstructive pulmonary disease (COPD). In some embodiments, the subject is a mammal. In some embodiments, the subject is a human. In some embodiments, the administering comprises inhalation by nebulization.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A-1B illustrate rescue of function of CFTR using tRNA-LNP compositions of the present disclosure with a single administration.

FIG. 2A-2B illustrate rescue of function of CFTR using tRNA-LNP compositions of the present disclosure after repeat dosing.

FIG. 3A-3B illustrate rescue of function of CFTR using tRNA composition of the present disclosure over a time course study in G542X/F508del human bronchial epithelial cells (hBEs).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “disease,” as used herein, generally refers to an abnormal physiological condition that affects part or all of a subject, such as an illness (e.g., primary ciliary dyskinesia) or another abnormality that causes defects in the action of cilia in, for example, the lining the respiratory tract (lower and upper, sinuses, Eustachian tube, middle ear), in a variety of lung cells, in the fallopian tube, or flagella of sperm cells.

The term “polynucleotide” or “nucleic acid” as used herein generally refers to a polymeric form of nucleotides of any length, either ribonucleotides or deoxyribonucleotides, that comprise purine and pyrimidine bases, purine and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature.

The term “polypeptides,” as used herein, generally refers to polymer chains comprised of amino acid residue monomers which are joined together through amide bonds (peptide bonds). A polypeptide can be a chain of at least three amino acids, a protein, a recombinant protein, an antigen, an epitope, an enzyme, a receptor, or a structure analogue or combinations thereof. As used herein, the abbreviations for the L-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The term “engineered,” as used herein, generally refers to polynucleotides, vectors, and nucleic acid constructs that have been genetically designed and manipulated to provide a polynucleotide intracellularly. An engineered polynucleotide can be partially or fully synthesized in vitro. An engineered polynucleotide can also be cloned. An engineered polyribonucleotide can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long untranslated RNA (lncRNA), microRNA (miRNA), or another suitable RNA.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Example of lipid compositions include suspensions, emulsions, and vesicular compositions.

As used herein, the term “detectable” refers to an occurrence of, or a change in, a signal that is directly or indirectly detectable either by observation or by instrumentation. Typically, a detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density.

Unless otherwise indicated, all numbers expressing quantities, ranges, conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

As used herein, the term “ratio” generally refers to the relative amount of one or more molecules to another molecule(s). Non-limiting examples of the ratio(s) include molar ratio(s), weight ratio(s), or mass ratio(s).

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “————” represents an optional bond, which if present is either single or double. The symbol “—̅—̅—̅—̅” represents a single bond or a double bond. Thus, for example, the formula

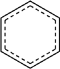

includes

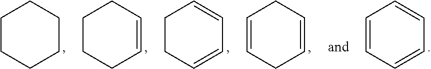

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

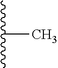

for methyl) indicates a point of attachment of the group. It is noted that the point of attachment is typically only identified in this manner for larger group in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

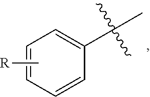

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

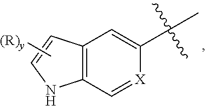

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

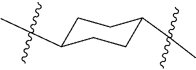 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C═CH, —C═CCH₃, and —CH₂C═CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

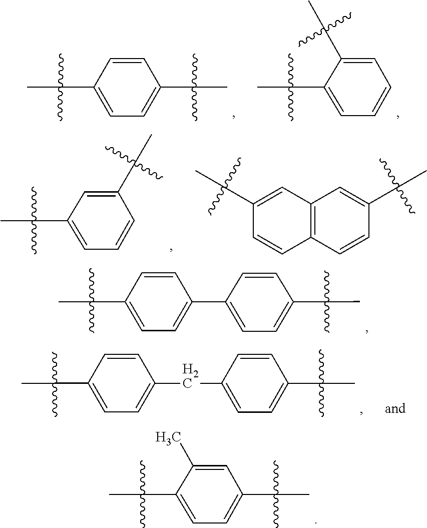

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OK or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

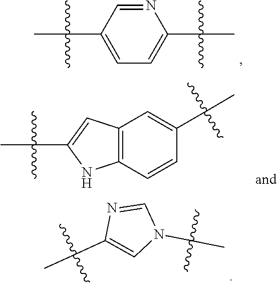

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

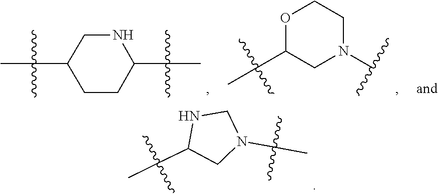 [SMILES: *C1CCC(*)NC1 ; *C1CN(*)CCO1 ; *C1CN(*)CN1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, funmaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, -[—CH₂CH₂-]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters.

Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “molar percentage” or “molar %” as used herein in connection with lipid composition(s) generally refers to the molar proportion of that component lipid relative to compared to all lipids formulated or present in the lipid composition.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### Cystic Fibrosis Transmembrane Conductance Regulator (CFTR)

Cystic fibrosis transmembrane conductance regulator (CFTR) is a membrane protein and chloride channel in vertebrates encoded by the CFTR gene. CFTR gene is on the long arm of chromosome 7, at position q31.2. Mutations of the CFTR gene affecting chloride ion channel function lead to dysregulation of epithelial fluid transport in the lung, pancreas and other organs, resulting in cystic fibrosis (CF).

Cystic fibrosis (CF) affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease. Complications of cystic fibrosis include thickened mucus in the lungs with frequent respiratory infections, and pancreatic insufficiency giving rise to malnutrition and diabetes. These conditions lead to chronic disability and reduced life expectancy. In male patients, the progressive obstruction and destruction of the developing vas deferens (spermatic cord) and epididymis appear to result from abnormal intraluminal secretions, causing congenital absence of the vas deferens and male infertility.

So far, nearly 1000 cystic fibrosis-causing mutations have been described. The vast majority of mutations are infrequent. The distribution and frequency of mutations varies among different populations. Mutations consist of replacements, duplications, deletions or shortenings in the CFTR gene. This may result in dysfunctional proteins which have less activity, are more quickly degraded or present in inadequate numbers. The most common mutation, DeltaF508 (ΔF508) results from a deletion (Δ) of three nucleotides which results in a loss of the amino acid phenylalanine (F) at the 508th position on the protein. As a result, the protein does not fold normally and is more quickly degraded.

### Compositions

In one aspect, the present disclosure provides a composition comprising a synthetic transfer ribonucleic acid (tRNA) as described herein assembled with a lipid composition as described herein. The lipid composition may comprise a zwitterionic lipid. The composition may be an aerosol composition. The composition may be formulated for aerosol administration.

Transfer Ribonucleic Acids (tRNAs)

As used herein, the term transfer RNA or tRNA refers to both traditional tRNA molecules as well as tRNA molecules with one or more modifications unless specifically noted otherwise. Transfer RNA is an RNA polymer that is about 70 to 100 nucleotides in length. During protein synthesis, a tRNA delivers an amino acid to the ribosome for addition to the growing peptide chain. Active tRNAs have a 3′CCA tail that may be transcribed into the tRNA during its synthesis or may be added later during post-transcriptional processing. The amino acid is covalently attached to the 2′ or 3′ hydroxyl group of the 3′-terminal ribose to form an aminoacyl-tRNA (aa-tRNA); an amino acid can spontaneously migrate from the 2′-OH to the 3′-OH and vice versa, but it is incorporated into a growing protein chain at the ribosome from the 3′-OH position. A loop at the other end of the folded aa-tRNA molecule contains a sequence of three bases known as the anticodon. When this anticodon sequence base-pairs with a three-base codon sequence in a ribosome-bound messenger RNA (mRNA), the aa-tRNA binds to the ribosome and its amino acid is incorporated into the nascent protein chain. Since all tRNAs that base-pair with a specific codon are aminoacylated with a single specific amino acid, the translation of the genetic code is effected by tRNAs: each of the 61 non-termination codons in an mRNA directs the binding of its cognate aa-tRNA and the addition of a single specific amino acid to the growing protein polymer. In some embodiments, the tRNA may comprise a sequence in the anticodon region of the tRNA such that the aa-tRNA base-pairs with a different codon on the mRNA. In certain embodiments, the mutated tRNA introduces a different amino acid into the growing protein chain than the amino acid encoded by the mRNA. In other embodiments, the mutated tRNA base-pairs with a stop codon and introduces an amino acid instead of terminating protein synthesis, thereby allowing the nascent peptide to continue to grow. In some embodiments, a tRNA, wild-type or mutated, may read through a stop codon and introduce an amino acid instead of terminating protein synthesis. In some embodiments, the tRNA may comprise a full-length tRNA with the 3′-terminal-CCA nucleotides included. In other embodiments, tRNAs lacking the 3′-terminal -A, -CA, or CCA are made full-length in vivo by the CCA-adding enzyme.

In other aspects, the present compositions may further comprise one or more modified tRNA molecules including: acylated tRNA; alkylated tRNA; a tRNA containing one or more bases other than adenine, cytosine, guanine, or uracil; a tRNA covalently modified by the attachment of a specific ligand or antigenic, fluorescent, affinity, reactive, spectral, or other probe moiety; a tRNA containing one or more ribose moieties that are methylated or otherwise modified; aa-tRNAs that are aminoacylated with an amino acid other than the 20 natural amino acids, including non-natural amino acids that function as a carrier for reagents, specific ligands, or as an antigentic, fluorescent, reactive, affinity, spectral, or other probe; or any combination of these compositions. Some examples of modified tRNA molecules are taught by Söll, et al., 1995; El Yacoubi, et al., 2012; Grosjean and Benne, et al., 1998; Hendrickson, et al., 2004; Ibba and Söll, 2000; Johnson, et al., 1995; Johnson, et al., 1982; Crowley, et al., 1994; Beier and Grimm, 2001; Tones, et al., 2014; and Björk, et al., 1987, all of which are incorporated herein by reference.

In some embodiments, the synthetic tRNA is a folded tRNA. The folded tRNA may be folded such that the tRNA may perform a function. For example, folded tRNA may comprise a folded shape that allows for recognition of a codon or allow for the loading of an amino acid. The folded tRNA may perform a function that an unfolded tRNA may be unable to perform. The folded tRNA may comprise a T-arm, a D-arm, an anticodon arm, a variable loop, an acceptor stem, or a combination thereof. The folded tRNA may comprise motifs, structures, or sequences that may perform a particular function. The synthetic tRNA may comprise an anticodon arm that is configured to recognize a premature stop codon. The synthetic tRNA may comprise an anticodon arm that may recognize a codon that may normally code for an amino acid or stop codon, and the acceptor stem may be configured to be operably linked to a non-corresponding amino acid or stop codon. For example, the synthetic tRNA may comprise an anticodon arm that may recognize a stop codon, and the acceptor stem may be configured to be operably linked to an amino acid. This may allow the tRNA to recognize a premature stop codon and, instead of causing or allowing the termination of translation, may add an amino acid to the polypeptide chain. This may allow the tRNA to prevent a premature termination of a polypeptide translation.

In some embodiments, the synthetic tRNA comprises an acceptor stem that is configured to be operably linked to an amino acid. The synthetic tRNA may comprise an acceptor stem that is configured to be operably linked to an arginine. The synthetic tRNA may comprise an acceptor stem that is configured to be operably linked to a glycine. The synthetic tRNA may comprise an acceptor stem that is configured to be operably linked to an alanine. The synthetic tRNA may comprise an acceptor stem that is configured to be operably linked to a cysteine. The synthetic tRNA may comprise an acceptor stem that is configured to be operably linked to an aspartic acid. The synthetic tRNA may comprise an acceptor stem that is configured to be operably linked to a glutamic acid. The synthetic tRNA may comprise an acceptor stem that is configured to be operably linked to a phenylalanine. The synthetic tRNA may comprise an acceptor stem that is configured to be operably linked to a histidine. The synthetic tRNA may comprise an acceptor stem that is configured to be operably linked to an isoleucine. The synthetic tRNA may comprise an acceptor stem that is configured to be operably linked to a lysine. The synthetic tRNA may comprise an acceptor stem that is configured to be operably linked to a leucine. The synthetic tRNA may comprise an acceptor stem that is configured to be operably linked to a methionine. The synthetic tRNA may comprise an acceptor stem that is configured to be operably linked to an asparagine. The synthetic tRNA may comprise an acceptor stem that is configured to be operably linked to a proline. The synthetic tRNA may comprise an acceptor stem that is configured to be operably linked to a glutamine. The synthetic tRNA may comprise an acceptor stem that is configured to be operably linked to a serine. The synthetic tRNA may comprise an acceptor stem that is configured to be operably linked to a threonine. The synthetic tRNA may comprise an acceptor stem that is configured to be operably linked to a valine. The synthetic tRNA may comprise an acceptor stem that is configured to be operably linked to a tryptophan. The synthetic tRNA may comprise an acceptor stem that is configured to be operably linked to a tyrosine.

In some embodiments, the tRNA is a tRNA amber suppressor. In other embodiments, the tRNA is a tRNA opal suppressor. In other embodiments, the tRNA is a tRNA ochre suppressor. In some embodiments, the tRNA is a tRNA frameshift suppressor.

In some embodiments, the synthetic tRNA comprises a nucleic acid sequence selected from SEQ ID NOs: 1-20. In some embodiments, the synthetic tRNA comprises a nucleic acid sequence having at least about 80% identity to a sequence selected from SEQ ID NOs: 1-20. In some embodiments, the synthetic tRNA comprises a nucleic acid sequence having at least about 85% identity to a sequence selected from SEQ ID NOs: 1-20. In some embodiments, the synthetic tRNA comprises a nucleic acid sequence having at least about 86%, 87%, 88%, 89%, 90%, 91%, 92%, 93%, 94%, 95%, %%, 97%, 98%, or 99%, or more, identity to a sequence selected from SEQ ID NOs: 1-20. In some embodiments, the synthetic tRNA comprises a nucleic acid sequence that is identical to a sequence selected from SEQ ID NOs: 1-20.

TABLE 1
Example tRNA sequences

| SEQ | |
|---|---|
| ID NO: | Sequence |
| 1 | 5′- |
| | GGCCGCGTGGCCTAATGGAtAAGGCGTCTGACTTCAGATCAGAAGAtTGCAGGTTCGAGTCCTGCCGC |
| | GGTCGCCA-3′ |
| 2 | 5′- |
| | GGCCGCGUGGCCUAAUGGAUAAGGCGUCUGACUUCAGAUCAGAAGAUUGCAGGUUCGAGUCCUGCCGC |
| | GGUCGCCA-3′ |
| 3 | 5′- |
| | GGCCUCGUGGCGCAACGGUAGCGCGUCUGACUUCAGAUCAGAAGGUUGCGUGUUCAAAUCACGUCGGG |
| | GUCACCA-3′ |
| 4 | 5′- |
| | GCGUUGGUGGUAUAGUGGUUAGCAUAGCUGCCUUCAAAGCAGUUGACCCGGGUUCGAUUCCCGGCCAA |
| | CGCACCA-3′ |
| 5 | 5′- |
| | GCAGCGAUGGCCGAGUGGUUAAGGCGUUGGACUUCAAAUCCAAUGGGGUCUCCCCGCGCAGGUUCGAA |
| | CCCUGCUCGCUGCGCCA-3′ |
| 6 | 5′- |
| | GGUCCCAUGGUGUAAUGGUUAGCACUCUGGACUUUAAAUCCAGCGAUCCGAGUUCAAAUCUCGGUGGG |
| | ACCUCCA-3′ |
| 7 | 5′- |
| | GCCCGGAUAGCUCAGUCGGUAGAGCAUCAGACUUUAAAUCUGAGGGUCCAGGGUUCAAGUCCCUGUUC |
| | GGGCGCCA-3′ |
| 8 | 5′- |
| | ACCAGGAUGGCCGAGUGGUUAAGGCGUUGGACUUUAGAUCCAAUGGACAUAUGUCCGCGUGGGUUCGA |
| | ACCCCACUCCUGGUACCA-3′ |
| 9 | 5′- |
| | GCCCGGCUAGCUCAGUCGGUAGAGCAUGGGACUCUAAAUCCCAGGGUCGUGGGUUCGAGCCCCACGUU |
| | GGGCGCCA-3′ |
| 10 | 5′- |
| | UCCCUGGUGGUCUAGUGGUUAGGAUUCGGCGCUAUCACCGCCGCGGCCCGGGUUCGAUUCCCGGUCAG |
| | GGAACCA-3′ |
| 11 | 5′- |
| | UCCCUGGUGGUCUAGUGGUUAGGAUUCGGCGCUAUCACCGCCGCGGCCCGGGUUCGAUUCCCGGUCAG |
| | GGAACCA-3′ |
| 12 | 5′- |
| | GUCAGGAUGGCCGAGUGGUCUAAGGCGCCAGACUCUAGUUCUGGUCUCCGUAUGGAGGCGUGGGUUCG |
| | AAUCCCACUUCUGACACCA-3′ |
| 13 | 5′- |
| | ACCAGGAUGGCCGAGUGGUUAAGGCGUUGGACUUCAGAUCCAAUGGACAUAUGUCCGCGUGGGUUCGA |
| | ACCCCACUCCUGGUACCA-3′ |
| 14 | 5′- |
| | CCUUCAAUAGUUCAGCUGGUAGAGCAGAGGACUCUAGGUCCUUAGGUUGCUGGUUCGAUUCCAGCUUG |
| | AAGGACCA-3′ |
| 15 | 5′- |
| | CCUUCAAUAGUUCAGCUGGUAGAGCAGAGGACUUUAGGUCCUUAGGUUGCUGGUUCGAUUCCAGCUUG |
| | AAGGACCA-3′ |
| 16 | 5′- |
| | GGCCUCGUGGCGCAACGGUAGCGCGUCUGACUCUAGAUCAGAAGGUUGCGUGUUCAAAUCACGUCGGG |
| | GUCACCA-3′ |
| 17 | 5′- |
| | GCUGUGAUGGCCGAGUGGUUAAGGCGUUGGACUCGAAAUCCAAUGGGGUCUCCCCGCGCAGGUUCGAA |
| | UCCUGCUCACAGCGCCA-3′ |
| 18 | 5′- |
| | GCAGCGAUGGCCGAGUGGUUAAGGCGUUGGACUUUAAAUCCAAUGGGGUCUCCCCGCGCAGGUUCGAA |
| | CCCUGCUCGCUGCGCCA-3′ |
| 19 | 5′- |
| | GCGUUGGUGGUAUAGUGGUGAGCAUAGCUGCCUUCAAAGCAGUUGACCCGGGUUCGAUUCCCGGCCAA |
| | CGCACCA-3′ |
| 20 | 5′- |
| | GCGUUGGUGGUAUAGUGGUAAGCAUAGCUGCCUUCAAAGCAGUUGACCCGGGUUCGAUUCCCGGCCAA |
| | CGCACCA-3′ |

### Lipid Compositions

### Zwiiterionic Lipids

In some embodiments, the lipid composition of the present application comprises the zwitterionic lipid at a molar percentage of about 1% to about 60% In some embodiments, the lipid composition comprises the zwitterionic lipid at a molar percentage of at least (about) 1%, at least (about) 5%, at least (about) 10%, at least (about) 15%, at least (about) 20%, at least (about) 25%, at least (about) 30%, at least (about) 35%, at least (about) 40%, at least (about) 45%, at least (about) 50%, at least (about) 55%, or at least (about) 60%. In some embodiments, the lipid composition comprises the zwitterionic lipid at a molar percentage of at most (about) 60%, at most (about) 55%, at most (about) 50%, at most (about) 45%, at most (about) 40%, at most (about) 35%, at most (about) 30%, at most (about) 25%, at most (about) 20%, at least (about) 15%, at most (about) 10%, or at most (about) 5%. In some embodiments, the lipid composition comprises the zwitterionic lipid at a molar percentage of (about) 1%, (about) 2%, (about) 5%, (about) 10%, (about) 15%, (about) 20%, (about) 25%, (about) 30%, (about) 35%, (about) 40%, (about) 45%, (about) 50%, (about) 55%, or (about) 60%, or a range between any two of the foregoing values.

In some embodiments of the lipid composition, the zwitterionic lipid is a zwitterionic phospholipid.

In some embodiments of the lipid composition, the zwitterionic lipid comprises a sulfonate anion. The zwitterionic lipid may further comprise a quaternary ammonium cation.

In some embodiments, the zwitterionic lipid has a structural formula (I):

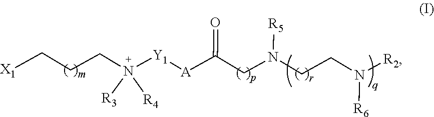

or a pharmaceutically acceptable salt thereof, wherein:
X₁ is —S(O)₂O⁻, or —OP(O)ORₑO⁻, wherein:
   R_{c} is hydrogen, alkyl_{(C≤6)}, or substituted alkyl_{(C≤6)};
Y₁ is alkanediyl_{(C≤12)}, alkenediyl_{(C≤12)}, or a substituted version thereof;
A is —NRₐ—, —S—, or —O—;
Rₐ, R₃ and R₄ are each independently hydrogen, alkyl_{(C≤6)}, or substituted alkyl_{(C≤6)}; or
alternatively, Rₐ is taken together with R₃ or R₄ to form alkanediyl_{(C≤8)} or substituted alkanediyl_{(C≤8)};
R₂ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
R₅ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
R₆ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
wherein:
   R′ and R″ are each independently hydrogen, alkyl_{(C≤8)}, substituted alkyl_{(C≤8)}, or —Z₂A′R₇,
   wherein:
      Z₂ is alkanediyl_{(C≤4)} or substituted alkanediyl_{(C≤4)};
      A′ is —CHRⱼ—, —C(O)O—, or —C(O)NR_{b}—, wherein:
         R_{b} is hydrogen, alkyl_{(C≤6)}, or substituted alkyl_{(C≤6)}; and
         Rⱼ is hydrogen, halo, hydroxy, acyloxy_{(C≤24)}, or substituted acyloxy_{(C≤24)};
      R₇ is alkyl_{(C6-24)}, substituted alkyl_{(C6-24)}, alkenyl_{(C6-24)}, or substituted alkenyl_{(C6-24)};
   Z₃ is alkanediyl_{(C≤4)} or substituted alkanediyl_{(C≤4)};
   A″ is —CHRₖ—, —C(O)O—, or —C(O)NRₗ—;
      Rₗ is hydrogen, alkyl_{(C≤6)}, or substituted alkyl_{(C≤6)}; and
      Rₖ is hydrogen, halo, hydroxy, acyloxy_{(C≤24)}, or substituted acyloxy_{(C≤24)}; and
   R₈ is alkyl_{(C6-24)}, substituted alkyl_{(C6-24)}, alkenyl_{(C6-24)}, or substituted alkenyl_{(C6-24)};
q is 1 or 2;
r is 1, 2, or 3; and
m and p are each independently 0, 1, 2, or 3.

In some embodiments of the zwitterionic lipid of Formula (I), X, is —S(O)₂O—.

In some embodiments of the zwitterionic lipid of Formula (I), Y₁ is alkanediyl_{(C≤12)} or alkenediyl_{(C≤12)}. In some embodiments, A is —NRₐ—. In some embodiments, Rₐ is hydrogen, alkyl_{(C≤6)}, or substituted alkyl_{(C≤6)}.

In some embodiments of the zwitterionic lipid of Formula (I), R₃ is hydrogen, alkyl_{(C≤6)}, or substituted alkyl_{(C≤6)}.

In some embodiments of the zwitterionic lipid of Formula (I), R₄ is hydrogen, alkyl_{(C≤6)}, or substituted alkyl_{(C≤6)}.

In some embodiments of the zwitterionic lipid of Formula (I), R₂ is hydrogen, alkyl_{(C≤8)}, substituted alkyl_{(C≤8)}, or —Z₃A″R₈. In some embodiments of R₂, Z₃ is alkanediyl_{(C≤4)}; A″ is —CHRₖ—, —C(O)O—, or —C(O)NRₗ—, wherein: Rₗ is hydrogen, alkyl_{(C≤6)}, or substituted alky_{(C≤6)}; and Rₖ is hydrogen, halo, hydroxy, acyloxy_{(C≤24)}, or substituted acyloxy_{(C≤24)}; and R₈ is alkyl_{(C6-24)} or alkenyl_{(C6-24)}.

In some embodiments of the zwitterionic lipid of Formula (I), R₅ is hydrogen, alkyl_{(C≤8)}, substituted alkyl_{(C≤8)}, or —Z₃A″R₈. In some embodiments of R₅, Z₃ is alkanediyl_{(C≤4)}; A″ is —CHRₖ—, —C(O)O—, or —C(O)NRₗ—, wherein: Rₗ is hydrogen, alkyl_{(C≤6)}, or substituted alkyl_{(C≤6)}; and Rₖ is hydrogen, halo, hydroxy, acyloxy_{(C≤24)}, or substituted acyloxy_{(C≤24)}; and R₈ is alkyl_{(C6-24)} or alkenyl_{(C6-24)}.

In some embodiments of the zwitterionic lipid of Formula (I), R₆ is alky_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, or a substituted version of any of these groups. In some embodiments, R₆ is alkyl_{(C≤8)} or substituted alkyl_{(C≤8)}. In some embodiments, R₆ is -alkanediyl_{(C≤6)}—NR′R″. In some embodiments of R₆, R′ is —Z₂A′R₇. In some embodiments, Z₂ is alkanediyl_{(C≤4)}; A′ is —CHRⱼ—, —C(O)O—, or —C(O)NR_{b}—, wherein: R_{b} is hydrogen, alkyl_{(C≤6)}, or substituted alkyl_{(C≤6)} and R, is hydrogen, halo, hydroxy, acyloxy_{(C≤24)}, or substituted acyloxy_{(C≤24)}; R₇ is alkyl_{(C6-24)} or alkenyl_{(C6-24)}. In some embodiments, R″ is —Z₂A′R₇. In some embodiments of R″, Z₂ is alkanediyl_{(C≤4)}; A′ is —CHRⱼ—, —C(O)O—, or —C(O)NR_{b}—, wherein: R_{b} is hydrogen, alkyl_{(C≤6)}, or substituted alkyl_{(C≤6)} and Rⱼ is hydrogen, halo, hydroxy, acyloxy_{(C≤24)}, or substituted acyloxy_{(C≤24)}; R₇ is alkyl_{(C6-24)} or alkenyl_{(C6-24)}.

In some embodiments of the zwitterionic lipid of Formula (I), q is 2.

In some embodiments of the zwitterionic lipid of Formula (I), r is 2 or 3.

In some embodiments, the zwitterionic lipid has a structural formula selected from the group consisting of:

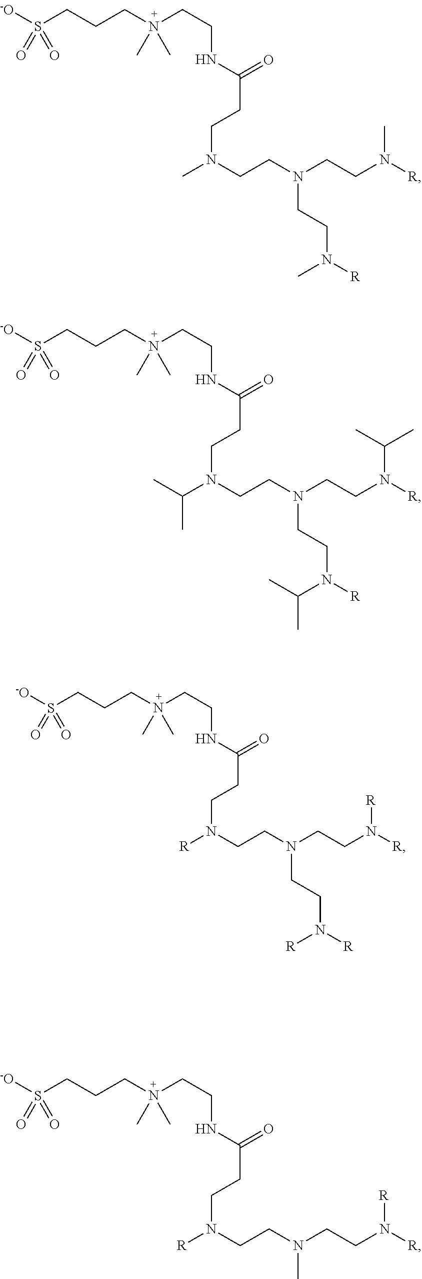

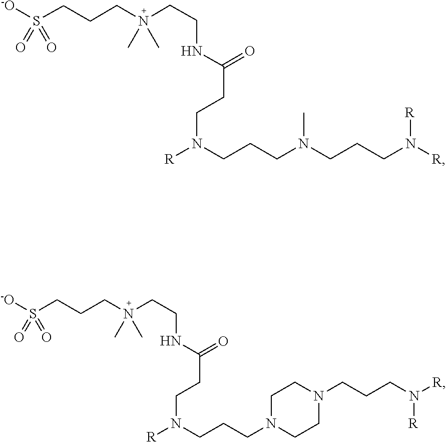

and pharmaceutically acceptable salts thereof, wherein: R is selected from the group consisting of H, —CH₂CH(OH)R₈, —CH₂CH₂C(O)OR₈, and —CH₂CH₂C(O)NHR₈, wherein: R₈ is selected from the group consisting of octyl, decyl, dodecyl, tetradecyl, hexadecyl, and octadecyl.

In some embodiments, the zwitterionic lipid has a structural formula:

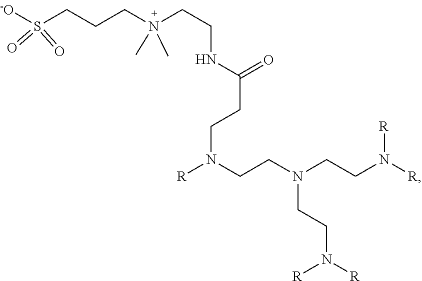

or a pharmaceutically acceptable salt thereof.

In some embodiments, the zwitterionic lipid is a compound selected from the group consisting of:

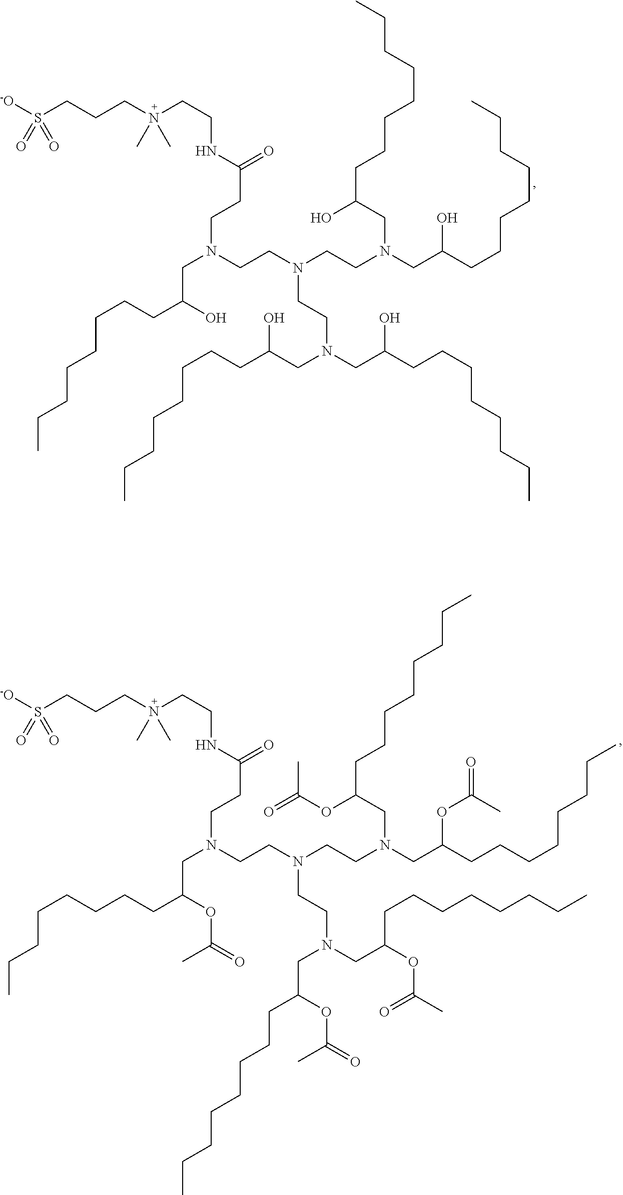

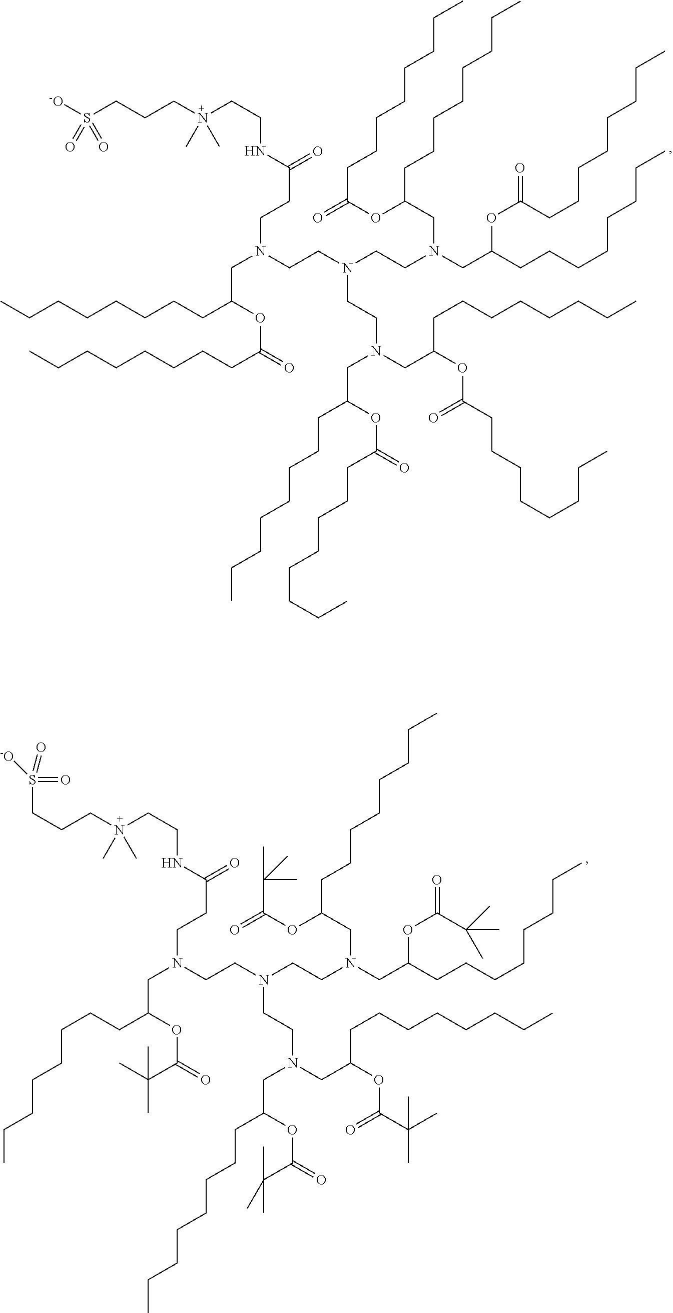

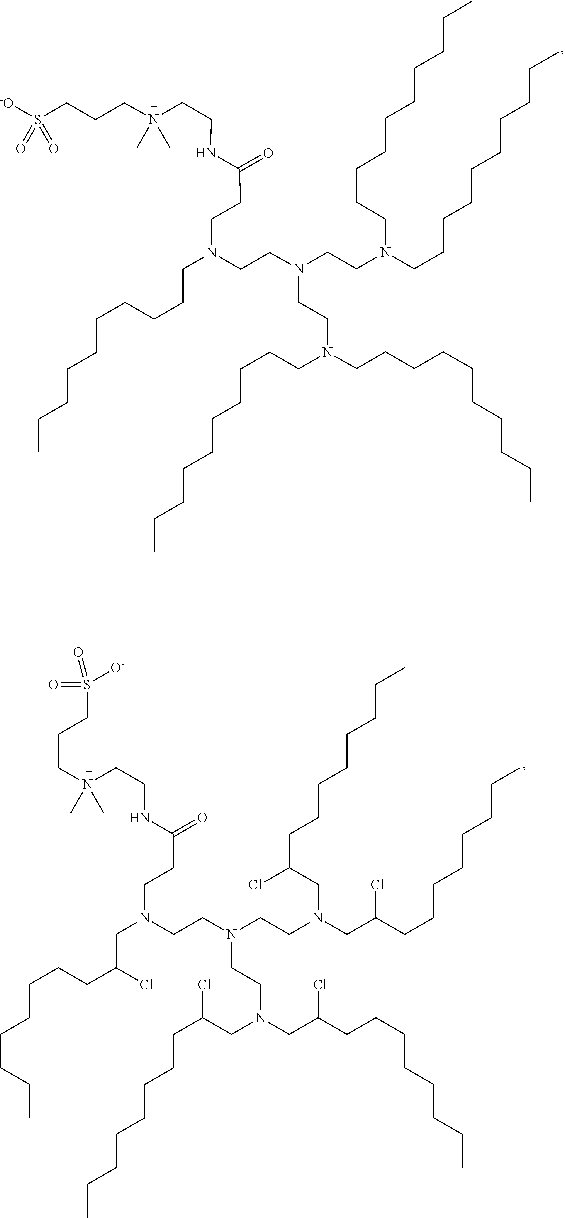

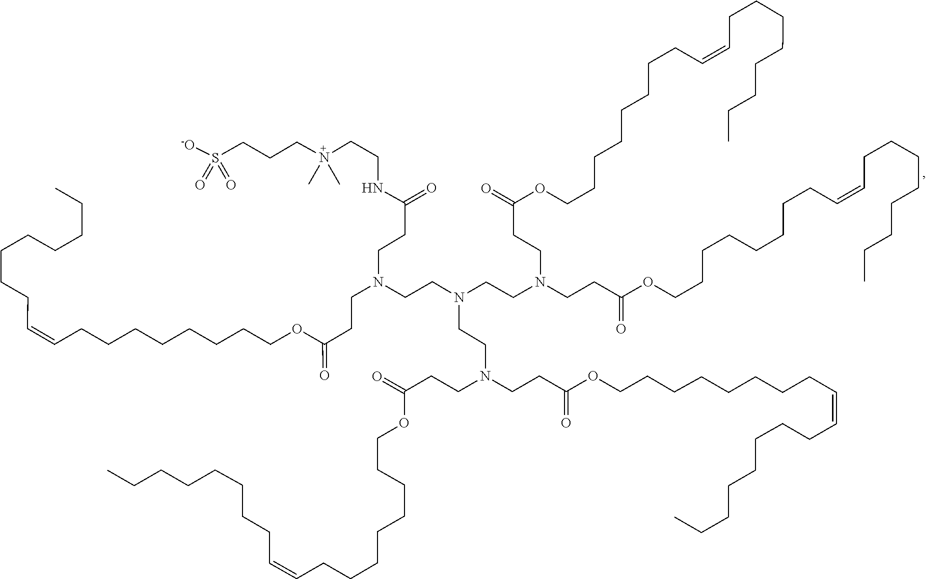

and pharmaceutically acceptable salts thereof.

In some embodiments of the lipid composition, the zwitterionic lipid comprises an alkylated or alkenylated phosphate anion. The alkylated or alkenylated phosphate anion may have a structural formula

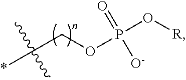

where R may alkyl or alkenyl; n is 1, 2, 3, 4, 5, or 6; and * may indicate a point of attachment of the alkylated or alkenylated phosphate anion, optionally, * may indicate a point of attachment of the alkylated or alkenylated phosphate anion to a quaternary ammonium cation.

In some embodiments, the lipid composition comprises more than one zwitterionic lipids comprising the zwitterionic lipid and a second zwitterionic lipid separate from the (first) zwitterionic lipid. The second zwitterionic lipid may be a phospholipid.

In some embodiments, the (e.g., first or second) zwitterionic lipid or phospholipid comprises one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the (e.g., first or second) zwitterionic lipid or phospholipid is a phosphatidylcholine. In some embodiments, the (e.g., first or second) zwitterionic lipid or phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments, the more than one zwitterionic lipids are present in the lipid composition at a molar percentage of about 1% to about 60% In some embodiments, the more than one zwitterionic lipids are present in the lipid composition at a molar percentage of at least (about) 5%, at least (about) 10%, at least (about) 15%, at least (about) 20%, at least (about) 25%, at least (about) 30%, at least (about) 35%, at least (about) 40%, at least (about) 45%, at least (about) 50%, at least (about) 55%, or at least (about) 60%. In some embodiments, the more than one zwitterionic lipids are present in the lipid composition at a molar percentage of at most (about) 60%, at most (about) 55%, at most (about) 50%, at most (about) 45%, at most (about) 40%, at most (about) 35%, at most (about) 30%, at most (about) 25%, at most (about) 20%, at least (about) 15%, at most (about) 10%, or at most (about) 5%. In some embodiments, the more than one zwitterionic lipids are present in the lipid composition at a molar percentage of (about) 5%, (about) 10%, (about) 15%, (about) 20%, (about) 25%, (about) 30%, (about) 35%, (about) 40%, (about) 45%, (about) 50%, (about) 55%, or (about) 60%, or a range between any two of the foregoing values.

In some embodiments, a (e.g., weight or mass) ratio of the zwitterionic lipid to the synthetic tRNA is of no more than about 50:1, 40:1, 30:1, 20:1, 10:1, 7.5:1, or 5:1. In some embodiments, a (e.g., weight or mass) ratio of the zwitterionic lipid to the synthetic tRNA is of at least about 1:1, or 2:1. In some embodiments, a (e.g., weight or mass) ratio of the zwitterionic lipid to the synthetic tRNA is of about 1:1 to about 50:1, or about 2:1 to about 50:1. In some embodiments, a (e.g., weight or mass) ratio of the zwitterionic lipid to the synthetic tRNA is of about 1:1 to about 40:1, or about 2:1 to about 40:1. In some embodiments, a (e.g., weight or mass) ratio of the zwitterionic lipid to the synthetic tRNA is of about 1:1 to about 30:1, or about 2:1 to about 30:1. In some embodiments, a (e.g., weight or mass) ratio of the zwitterionic lipid to the synthetic tRNA is of about 1:1 to about 20:1, or about 2:1 to about 20:1.

### Additional Lipids

In some embodiments of the lipid composition of the present application, the lipid composition further comprises an additional lipid including but not limited to a steroid or a steroid derivative, a polymer-conjugated lipid (e.g., polyethylene glycol (PEG)-conjugated lipid), or a combination thereof.

In some embodiments, a molar ratio of nitrogen in the lipid composition to phosphate in the synthetic polynucleotide (N/P ratio) is of no more than about 50:1, no more than about 40:1, no more than about 30:1, or no more than about 20:1. In some embodiments, a molar ratio of nitrogen in the lipid composition to phosphate in the synthetic polynucleotide (N/P ratio) is of at least about 1:1, at least about 2:1, at least about 3:1, at least about 4:1, or at least about 5:1. In some embodiments, a molar ratio of nitrogen in the lipid composition to phosphate in the synthetic polynucleotide (N/P ratio) is of about 1:1 to about 50:1, at least about 2:1 to about 50:1, at least about 3:1 to about 50:1, at least about 4:1 to about 50:1, or at least about 5:1 to about 50:1. In some embodiments, a molar ratio of nitrogen in the lipid composition to phosphate in the synthetic polynucleotide (N/P ratio) is of about 1:1 to about 40:1, at least about 2:1 to about 40:1, at least about 3:1 to about 40:1, at least about 4:1 to about 40:1, or at least about 5:1 to about 40:1. In some embodiments, a molar ratio of nitrogen in the lipid composition to phosphate in the synthetic polynucleotide (N/P ratio) is of about 1:1 to about 30:1, at least about 2:1 to about 30:1, at least about 3:1 to about 30:1, at least about 4:1 to about 30:1, or at least about 5:1 to about 30:1.

### Steroids or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

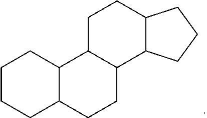 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

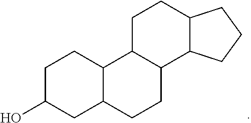 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

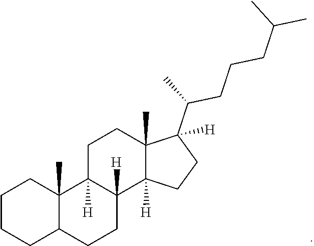

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar percentage of the steroid to the total lipid composition from about 40 to about 46. In some embodiments, the molar percentage is from about 40, 41, 42, 43, 44, 45, to about 46 or any range derivable therein. In other embodiments, the molar percentage of the steroid relative to the total lipid composition is from about 15 to about 40. In some embodiments, the molar percentage is 15, 16, 18, 20, 22, 24, 26, 28, 30, 32, 34, 36, 38, or 40, or any range derivable therein.

In some embodiments, the lipid composition comprises the steroid or steroid derivative at a molar percentage of about 1% to about 60%, about 5% to about 60%, about 10% to about 60%, or about 20% to about 60%.

In some embodiments of the lipid composition of the present application, the lipid composition comprises the steroid or steroid derivative at a molar percentage from about 15% to about 46%. In some embodiments of the lipid composition of the present application, the lipid composition comprises the steroid or steroid derivative at a molar percentage from about 20% to about 40%. In some embodiments of the lipid composition of the present application, the lipid composition comprises the steroid or steroid derivative at a molar percentage from about 25% to about 35%. In some embodiments of the lipid composition of the present application, the lipid composition comprises the steroid or steroid derivative at a molar percentage from about 30% to about 40%. In some embodiments of the lipid composition of the present application, the lipid composition comprises the steroid or steroid derivative at a molar percentage from about 20% to about 30%. In some embodiments of the lipid composition of the present application, the lipid composition comprises the steroid or steroid derivative at a molar percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, the lipid composition comprises the steroid or steroid derivative at a molar percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, PEG modified phosphatidylethanolamine (PE). In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

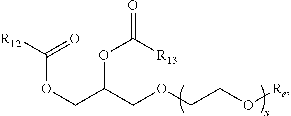

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

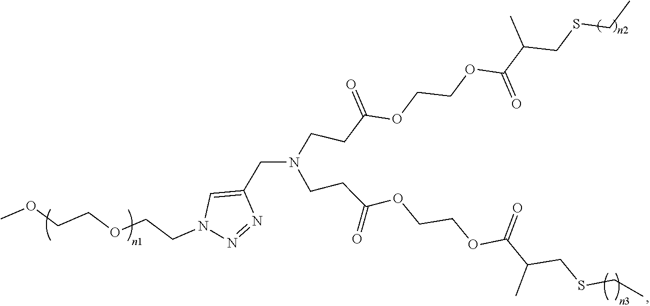

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar percentage of the PEG lipid to the total lipid composition from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

In some embodiments, the lipid composition comprises the polymer-conjugated lipid at a molar percentage of about 0.5% to about 12%. In some embodiments, the lipid composition comprises the polymer-conjugated lipid at a molar percentage of about 1% to about 12%. In some embodiments, the lipid composition comprises the polymer-conjugated lipid at a molar percentage of about 1.5% to about 12%.

In some embodiments of the lipid composition of the present application, the lipid composition comprises the polymer-conjugated lipid at a molar percentage from about 0.5% to about 10%. In some embodiments of the lipid composition of the present application, the lipid composition comprises the polymer-conjugated lipid at a molar percentage from about 1% to about 10%. In some embodiments of the lipid composition of the present application, the lipid composition comprises the polymer-conjugated lipid at a molar percentage from about 2% to about 10%. In some embodiments of the lipid composition of the present application, the lipid composition comprises the polymer-conjugated lipid at a molar percentage from about 3% to about 10%. In some embodiments of the lipid composition of the present application, the lipid composition comprises the polymer-conjugated lipid at a molar percentage from about 4% to about 10%. In some embodiments of the lipid composition of the present application, the lipid composition comprises the polymer-conjugated lipid at a molar percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, the lipid composition comprises the polymer-conjugated lipid at a molar percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Formulations

In some embodiments, where the composition is an aerosol composition or is formulated for aerosol administration, the composition has a droplet size from 0.5 micron (μm) to 10 μm. In some embodiments, the composition has a median droplet size from 0.5 μm to 10 μm. In some embodiments, the composition has an average droplet size from 0.5 μm to 10 μm. The droplet size may be determined by cascade impactor analysis or laser diffraction, or other suitable techniques for measuring aerosol droplets. The aerosol administration may be delivered to the respiratory epithelium.

In some embodiments of the composition, the composition can be formulated as any suitable dosage from known in the art. In some embodiments, the composition is formulated in a nanoparticle or a nanocapsule. In some embodiments, the composition is formulated for administration by any suitable route known in the art including, for example, oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments of the method, the composition of the present application is formulated for administration by a local rather than systemic manner, for example, via injection of the pharmaceutical composition directly into a targeted tissue, preferably in a sustained release formulation. Local delivery can be affected in various ways, depending on the tissue to be targeted.

In some embodiments of the method, aerosols containing the composition of the present application can be inhaled (for nasal, tracheal, or bronchial delivery). In some embodiments, the composition of the present application can be injected into the site of injury, disease manifestation, or pain, for example. In some embodiments, the composition of the present application can be provided in lozenges for oral, tracheal, or esophageal application. In some embodiments, the composition of the present application can be supplied in liquid, tablet or capsule form for administration to the stomach or intestines. In some embodiments, the composition of the present application can be supplied in suppository form for rectal or vaginal application. In some embodiments, the composition of the present application can even be delivered to the eye by use of creams, drops, or even injection.

### Kits

In one aspect, provided herein is a kit comprising the composition described herein. In some embodiments, the kit further comprises a container, and a label or package insert on or associated with the container.

### Methods

### Methods for Enhancing CFTR Expression or Activity in Cell(s)

In one aspect, provided herein is a method for enhancing an expression or activity of cystic fibrosis transmembrane conductance regulator (CFTR) protein in a cell. The method may comprise contacting the cell with a composition as described herein, e.g., comprising a transfer ribonucleic acid (tRNA) as described herein assembled with a lipid composition as described herein, to introduce an amino acid into a growing peptide chain of a CFTR protein in the cell, thereby yielding a therapeutically effective amount or activity of a functional variant of CFTR protein in the cell at least 24, 48, or 72 hours after contacting.

In one aspect, provided herein is a method for enhancing an expression or activity of cystic fibrosis transmembrane conductance regulator (CFTR) protein in a cell of a subject exhibiting or suspected of exhibiting a mutation in a CFTR gene. The method may comprise contacting the cell with a composition comprising a transfer ribonucleic acid (tRNA) assembled with a lipid composition to introduce an amino acid into a growing peptide chain of a CFTR protein in the cell at a position corresponding to the mutation in the CFTR gene of the subject, thereby yielding a therapeutically effective amount or activity of a functional variant of CFTR protein in the cell.

The therapeutically effective activity of the functional variant of CFTR protein may be determined by measuring a change in a transepithelial ion transport characteristic (e.g., transepithelial current or voltage) of a plurality of cells comprising the cell as compared to that of a reference plurality of cells, e.g., in absence of the contacting.

In some embodiments of the methods described herein, the contacting is repeated. The contacting may be repeated 1, 2, 3, or more times. In some embodiments, the contacting is at least once a week. In some embodiments, the contacting is at least twice a week. In some embodiments, the method yields a therapeutically effective amount or activity of a functional variant of CFTR protein in the cell at least 24 hours after each contacting. In some embodiments, a second contacting is performed, optionally at least about 1, 2, or 3 day(s) after the first contacting. In some embodiments, the method further comprises a third contacting wherein the third contacting is performed, optionally at least about 1, 2, or 3 day(s) after the second contacting. In some embodiments, the method yields a therapeutically effective amount or activity of a functional variant of CFTR protein in the cell at least 24 hours after a second contacting. In some embodiments, the method yields a therapeutically effective amount or activity of a functional variant of CFTR protein in the cell at least 24 hours after a third contacting. The composition in each contacting may be the same or identical. The therapeutically effective amount or activity of a functional variant of CFTR protein may increase after repeated contacting.

The contacting(s) may be performed in vivo. The contacting(s) may be performed in vitro. The contacting(s) may be performed ex vivo.

In some embodiments, the methods achieve a therapeutically effective activity of the functional variant of CFTR protein. In some embodiments, therapeutically effective activity may be measured by a transepithelial assay. The transepithelial assay may measure a voltage or a current which may correspond to the function of a functional protein. In some embodiments, the therapeutically effective activity of the functional variant of CFTR protein corresponds to a transepithelial current from about 2 micro-Ampere (μA) to about 30 ρA. In some embodiments, the therapeutically effective activity of the functional variant of CFTR protein corresponds to a transepithelial current of at least about 2 micro-Ampere (μA). In some embodiments, the therapeutically effective activity of the functional variant of CFTR protein corresponds to a transepithelial current of at least about 2 micro-Ampere (μA) per squared centimeter per minute (μA·cm⁻²·min⁻¹). In some embodiments, the therapeutically effective activity of the functional variant of CFTR protein corresponds to a transepithelial current from about 2 micro-Ampere (μA) per squared centimeter per minute (μA·cm⁻²·min⁻¹) to about 30 μA·cm⁻²·min⁻¹, The transepithelial current may be determined via the equivalent transepithelial current assay using the TECC24 system, such as those described elsewhere herein.

In some embodiments of the methods described herein, the method increases an amount of a functional variant of CFTR protein in the cell relative to a corresponding control. The functional variant may be a wild type CFTR protein. The functional variant may be a full length CFTR protein. In some embodiments, the method increases an amount of a functional variant CFTR protein in the cell relative to a corresponding control. In some embodiments, the control comprises a corresponding cell absent any one or more step(s) of the contacting(s). In some embodiments, the method increases an amount of the functional variant of CFTR protein by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3.0-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4.0-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, or at least about 5.0-fold, in the cell relative to a corresponding control.

In some embodiments, the method results in a therapeutically effective amount of the functional variant of CFTR protein in the cell. In some embodiments, the method results in a therapeutically effective amount of wild-type (WT) or full-length CFTR protein in the cell.

In some embodiment, the method enhances ion transport in the cell relative to a corresponding control. In some embodiment, the method enhances chloride transport in the cell relative to a corresponding control. In some embodiments, the control comprises a corresponding cell absent the contacting. In some embodiment, the method enhances ion transport by at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3.0-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4.0-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, or at least about 5.0-fold, in the cell relative to a corresponding control.

### Methods for Treating Cystic Fibrosis

In one aspect, provided herein is a method for treating a subject having or suspected of having a cystic fibrosis transmembrane conductance regulator (CFTR)-associated condition. The method may comprise administering to the subject a composition as described herein. In some embodiments, the CFTR-associated condition is cystic fibrosis, hereditary emphysema, or chronic obstructive pulmonary disease (COPD), or a combination thereof. The subject may be a mammal. The subject may be a human. In some embodiments, the administering comprises pulmonary administration. In some embodiments, the administering comprises inhalation by nebulization. In some embodiments, the administering comprises apical administration.

The methods of the disclosure may be able to treat a subject with cystic fibrosis based on properties of the formulation or compositions. Specifically the compositions described elsewhere herein may be able to penetrate the mucus associated with cystic fibrosis and thereby deliver the polynucleotides to the cells.

### Cell(s)

In some embodiments of the methods described herein, the cell is a lung cell. In some embodiments, the lung cell is a lung airway cell. Exemplary lung airway cell that can be targeted by the delivery of the present application includes but is not limited to basal cell, secretory cell such as goblet cell and club cell, ciliated cell, ionocyte and any combination thereof. In some embodiments of the method, the cell is an airway epithelial cell. In some embodiments, the cell is a bronchial epithelial cell. In some embodiments, the cell is an airway epithelial cell. In some embodiments, the cell is a basal cell characterized by expression of p63 marker. In some embodiments, the cell is an ionocyte characterized by expression of FOXI1 marker. In some embodiments, the cell is undifferentiated. In some embodiments, the cell is differentiated. In some embodiments, the cell(s) is/are derived from the subject. The subject may be a mammal. The subject may be a human.

### Mutation(s)

In some embodiments, the cell or subject exhibits a mutation in CFTR gene or transcript. In some embodiments, the cell or subject exhibits a mutation in one or more of exons 11-27 of CFTR gene. the cell or subject exhibits a nonsense or frameshift mutation in one or more of exons 11-27 of CFTR gene. In some embodiments, the mutation is located at a position in the CFTR gene at which a change can give rise to a mutant protein having a mutation at F508, e.g., F508del. In some embodiments, the mutation is located at a position in the CFTR gene at which a change can give rise to a mutant protein having a mutation at R553, e.g., R553X, in the CFTR protein, which corresponds to c.1657C>T in the CFTR gene. In some embodiments, a cell or subject may have multiple mutations. In some embodiments, the mutation is associated with cystic fibrosis, hereditary emphysema, or chronic obstructive pulmonary disease (COPD).

### EXAMPLES

### Example 1. Generation of tRNA

To generate tRNA, DNA fragments encoding a specific tRNA sequence behind a T7 RNA polymerase promoter sequence were synthesized chemically. The 7 promoter-tRNA DNA sequences were amplified by PCR and then transcribed in vitro using 17 RNA polymerase using standard techniques such as those described by Green and Sambrook, 2012; Rio et al., 2011; Flanagan et al., 2003; and Janiak et al., 1992. The resulting tRNA transcripts were extracted with phenol, precipitated in high salt and ethanol, and purified by HPLC using a MonoQ ion exchange column. The purified tRNAs were precipitated, resuspended, and dialyzed into water.

### Example 2. Compensation of R553X/F508del CFTR Mutation by Suppressor tRNA LNP Formulations of the Present Application in Differentiated Primary hBE Cells from a R553X/F508del Subject

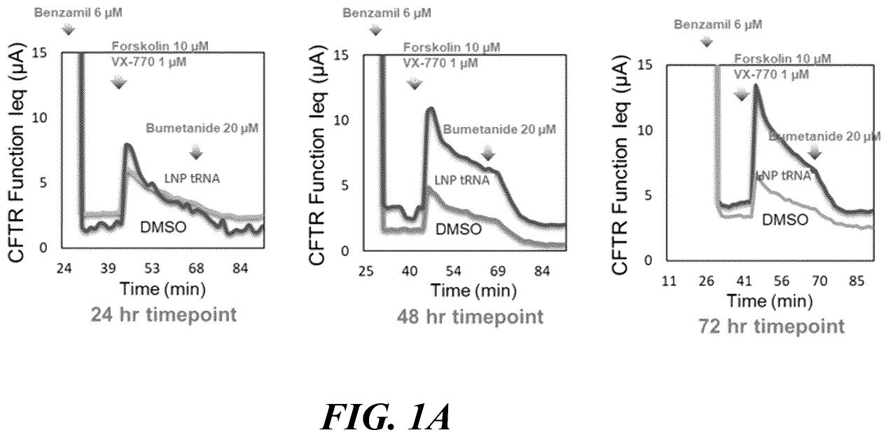

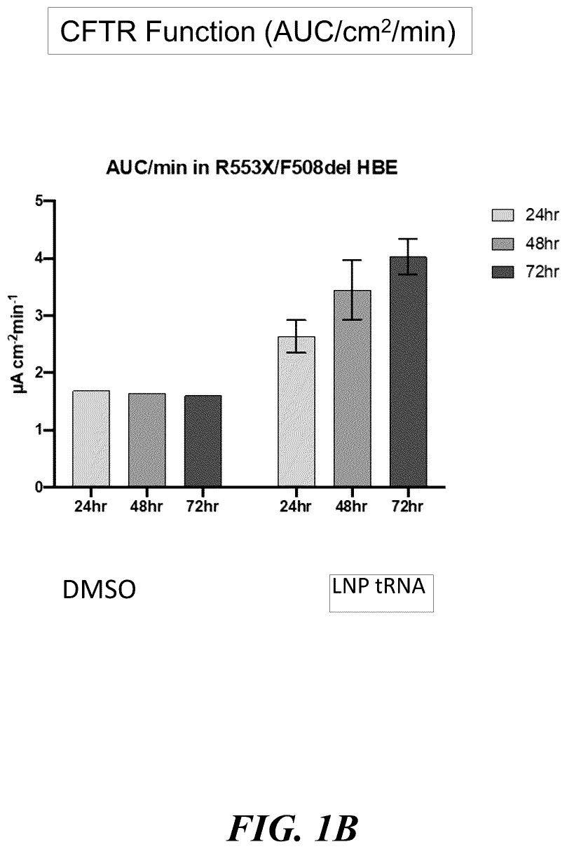

Suppressor tRNA encoding an arginine encapsulated by LNPs showed significant rescue of CFTR in the R553X/F508del CFTR hBE model. Briefly, a suppressor tRNA was encapsulated with an LNP composition and delivered to R553X/F508del CFTR hBE cells as apical liquid bolus or apical exposure of ALI hBE to nebulized LNPs aerosol. The hBE cell isolated from a cystic fibrosis patient with R553X/F508del CFTR genotype at passage 3 were seeded on 24 wells Transwell @ plates and airlifted after 96 hours. Cells grown following a 3 days/week feeding routine with Vertex ALI media. After 5 weeks hBE cell culture were considered as fully differentiated, polarized, and to be ready for the TECC24 functional assay. 4 days prior to treatment mucus was washed from the apical side of the hBE culture with 3 mM DTT in PBS. 24 hours before treatment cells were washed with PBS, additionally washed with PBS on the treatment day, treated apically with liquid bolus or VitroCell nebulized formulations, and tested after 24 or 24+n24 hour CO2 incubation as planned. Specifically, the hBE assay sequence includes background current/resistance recording interval (˜25 min), baseline Cl⁻ current recording interval (˜15 min) after inhibition of Na⁺ conductance with 6 μM Benzamil, 10 μM Forskolin+1 μM VX-770 induced CFTR activation interval (˜25 min), and 20 μM Bumetanide induced Cl⁻ current inhibition interval (˜25 min). The hBE transepithelial equivalent current traces [Ieq=Vt/(Rt−50), μA/cm] were reconstructed vs. time. The Forskolin/VX-770-induced Cl⁻ current responses were calculated as an Area Under the Ieq Curve (Ieq AUC) for time points between Forskolin/VX770 and INH-172 addition. The Ieq AUC/min values were statistically validated and compared across experimental samples. As shown in FIG. 1A, the apical bolus treatment with the LNPs comprising a suppressor tRNA recovered forskolin-dependent Cl⁻ current. in R553X/F508del hBEs, demonstrating rescue of CFTR function. The assay was run at 3 time points (24 hrs after administration, 48 hrs after administration, and 72 hrs after administration), all demonstrating the rescue of CFTR function. FIG. 1A and FIG. 1B show the function CFTR compared to cells treated with DMSO, demonstrating that the increase of CFTR function is significant compared against a negative control.

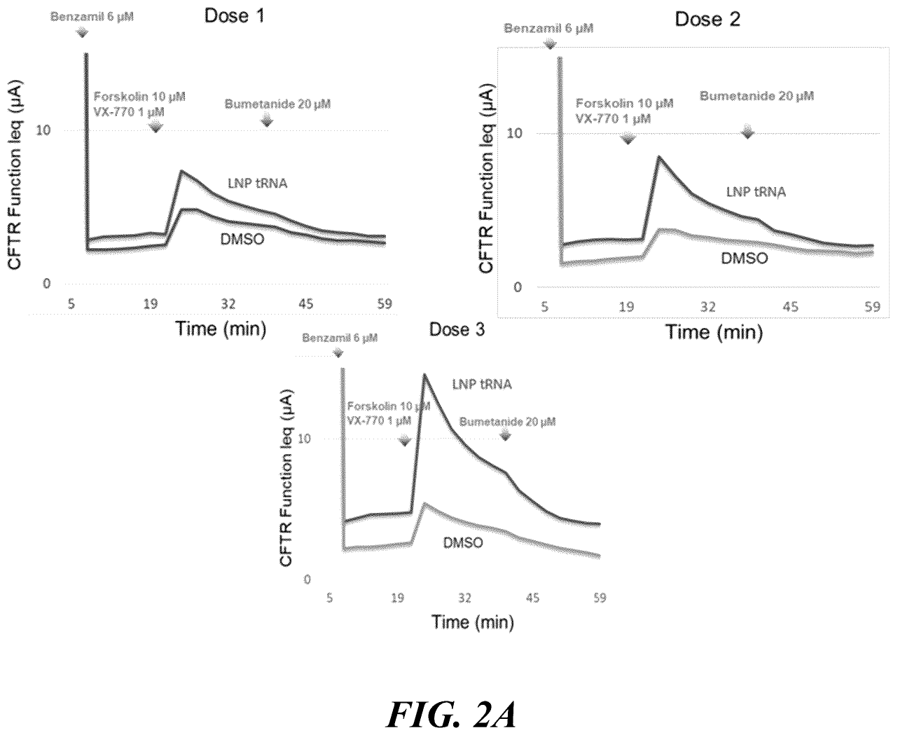

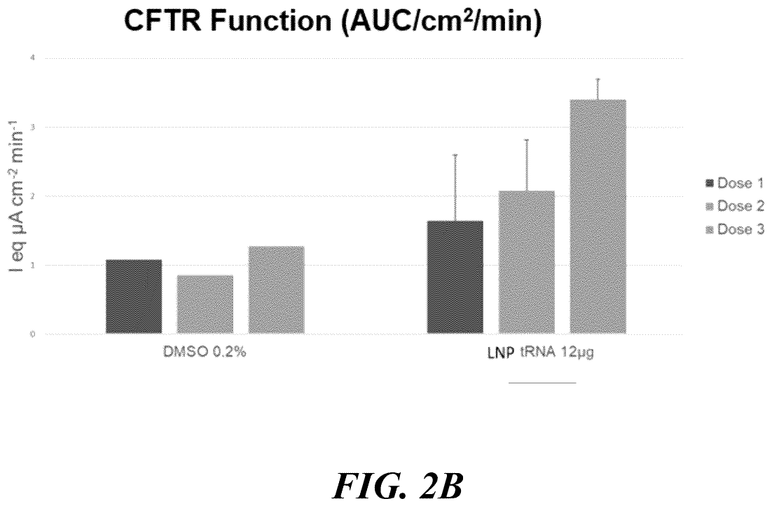

In a similar assay, the suppressor tRNA LNP formulation was repeatedly administered based on a twice a week dosing schedule. Using a similar protocol to determine the CFTR function, the repeated administrations showed improved CFTR function after each dose. FIG. 2A and FIG. 2B show that the first dose and second dose were able to improve CFTR function over a negative control, with the third dose increasing the function of CFTR even further.

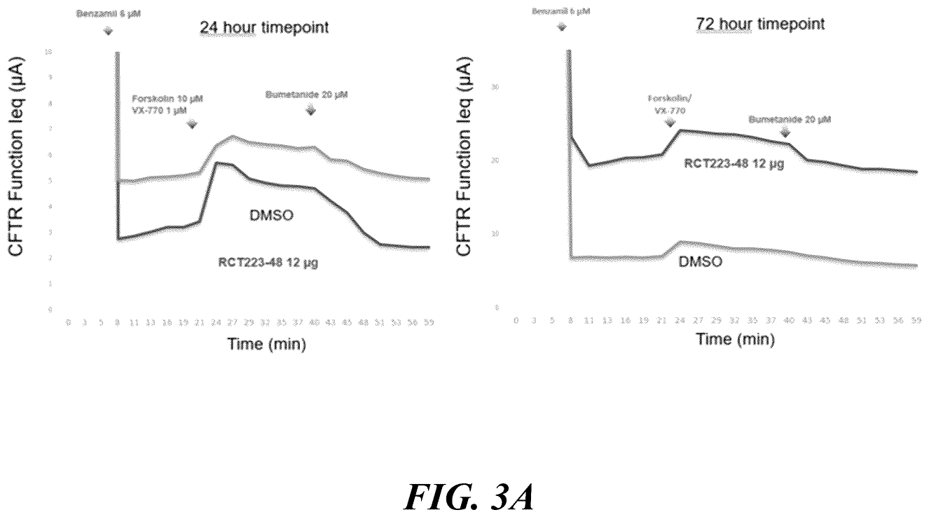

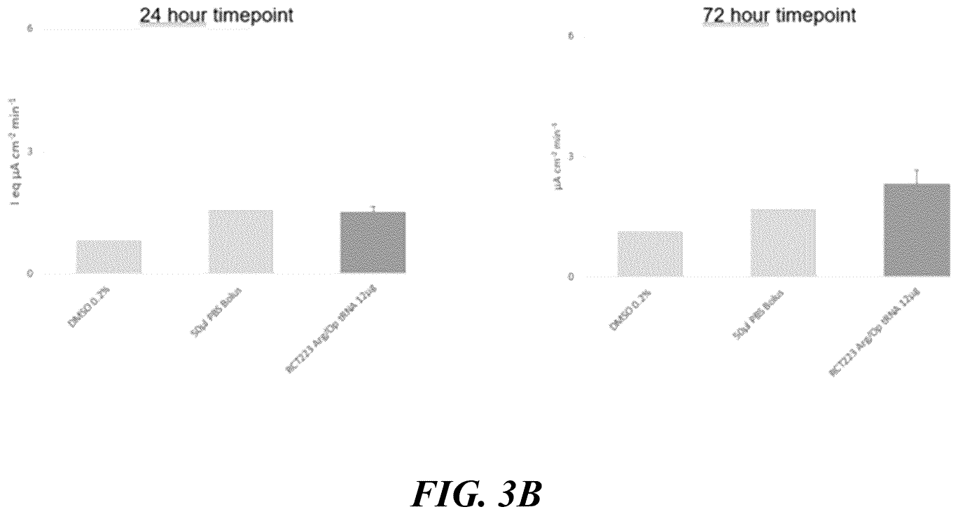

In a time course assay, the tRNA formulation (added as apical bolus formulation and as CFTR modulator to cell culture medium. The cell culture medium was changed after 24 hours. FIG. 3A and FIG. 3B show improved CFTR function at the 72-hour timepoint.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-65. (canceled)

66. A composition comprising a synthetic transfer ribonucleic acid (tRNA) assembled with a lipid composition, which lipid composition comprises a zwitterionic lipid, wherein said composition is formulated as an aerosol composition.

67. The composition of claim 66, wherein said composition has a droplet size from about 0.5 micron (μm) to about 10 μm, a median droplet size from about 0.5 μm to about 10 μm, an average droplet size from about 0.5 μm to about 10 μm, or any combination thereof.

68. The composition of claim 66, wherein said synthetic tRNA is a folded tRNA.

69. The composition of claim 68, wherein said folded tRNA comprises a T-arm, a D-arm, an anticodon arm, a variable loop, an acceptor stem, or a combination thereof.

70. The composition of claim 66, wherein said synthetic tRNA comprises an anticodon arm that is configured to recognize a premature stop codon.

71. The composition of claim 66, wherein said synthetic tRNA comprises an acceptor stem that is configured to couple to an arginine.

72. The composition of claim 66, wherein said synthetic tRNA comprises a polynucleotide sequence having at least about 80%, 85%, 86%, 87%, 88%, 89%, 90%, 91%, 92%, 93%, 94%, 95%, 96%, 97%, 98%, or 99% identity to a sequence selected from any one of SEQ ID NOs: 1-20.

73. The composition of claim 66, wherein a mass ratio of said zwitterionic amino lipid to said synthetic tRNA is of no more than about 50:1, 40:1, 30:1, or 20:1 or weight ratio of said zwitterionic amino lipid to said synthetic tRNA is of no more than about 50:1, 40:1, 30:1, or 20:1.

74. The composition of claim 66, wherein said lipid composition comprises said zwitterionic lipid at a molar percentage of about 1% to about 60%, wherein said molar percentage is determined based on the total lipids present in said lipid composition.

75. The composition of claim 66, wherein said lipid composition further comprises a steroid or steroid derivative, a polymer-conjugated lipid, or a combination thereof.

76. The composition of claim 75, wherein said lipid composition comprises said steroid or steroid derivative at a molar percentage of about 20% to about 60%, wherein said molar percentage is determined based on the total lipids present in said lipid composition.

77. The composition of claim 75, wherein said lipid composition comprises said polymer-conjugated lipid at a molar percentage of about 0.5% to about 12%, wherein said molar percentage is determined based on the total lipids present in said lipid composition.

78. The composition of claim 66, wherein a molar ratio of nitrogen molecules in said lipid composition to phosphate molecules in said synthetic tRNA (N/P ratio) is no more than about 50:1, 40:1, 30:1, 20:1, or 10:1.

79. The composition of claim 66, wherein the zwitterionic lipid has a structural formula (I): 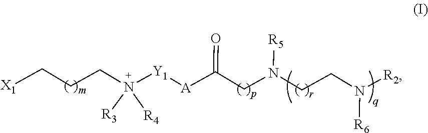
or a pharmaceutically acceptable salt thereof, wherein:
X₁ is —S(O)₂O⁻, or —OP(O)ORₑO⁻, wherein:
Rₑ is hydrogen, alkyl_{(C≤6)}, or substituted alkyl_{(C≤6)};
Y₁ is alkanediyl_{(C≤12)}, alkenediyl_{(C≤12)}, or a substituted version thereof;
A is —NRₐ—, —S—, or —O—;
Rₐ, R₃ and R₄ are each independently hydrogen, alkyl_{(C≤6)}, or substituted alkyl_{(C≤6)}; or alternatively, Rₐ is taken together with R₃ or R₄ to form alkanediyl_{(C≤8)} or substituted alkanediyl_{(C≤8)}; R₂ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
R₅ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
R₆ is selected from the group consisting of hydrogen, alkyl_{(C≤8)}, -alkanediyl_{(C≤6)}—NH₂, -alkanediyl_{(C≤6)}-alkylamino_{(C≤8)}, -alkanediyl_{(C≤6)}-dialkylamino_{(C≤12)}, -alkanediyl_{(C≤6)}—NR′R″, a substituted version of any of these groups, and —Z₃A″R₈;
wherein:
R′ and R″ are each independently hydrogen, alkyl_{(C≤8)}, substituted alkyl_{(C≤8)}, or —Z₂A′R₇,
wherein:
Z₂ is alkanediyl_{(C≤4)} or substituted alkanediyl_{(C≤4)};
A′ is —CHRⱼ—, —C(O)O—, or —C(O)NR_{b}—, wherein:
R_{b} is hydrogen, alkyl_{(C≤6)}, or substituted alkyl_{(C≤6)}; and
Rⱼ is hydrogen, halo, hydroxy, acyloxy_{(C≤24)}, or substituted acyloxy_{(C≤24)};
R₇ is alkyl_{(C6-24)}, substituted alkyl_{(C6-24)}, alkenyl_{(C6-24)}, or substituted alkenyl_{(C6-24)};
Z₃ is alkanediyl_{(C≤4)} or substituted alkanediyl_{(C≤4)};
A″ is —CHRₖ—, —C(O)O—, or —C(O)NRₗ—;
Rₗ is hydrogen, alkyl_{(C≤6)}, or substituted alkyl_{(C≤6)}; and
Rₖ is hydrogen, halo, hydroxy, acyloxy_{(C≤4)}, or substituted acyloxy_{(C≤24)}; and
R₈ is alkyl_{(C6-24)}, substituted alkyl_{(C6-24)}, alkenyl_{(C6-24)}, or substituted alkenyl_{(C6-24)};
q is 1 or 2;
r is 1, 2, or 3; and
m and p are each independently 0, 1, 2, or 3.

80. The composition of claim 66, wherein the zwitterionic lipid has a structural formula selected from the group consisting of: 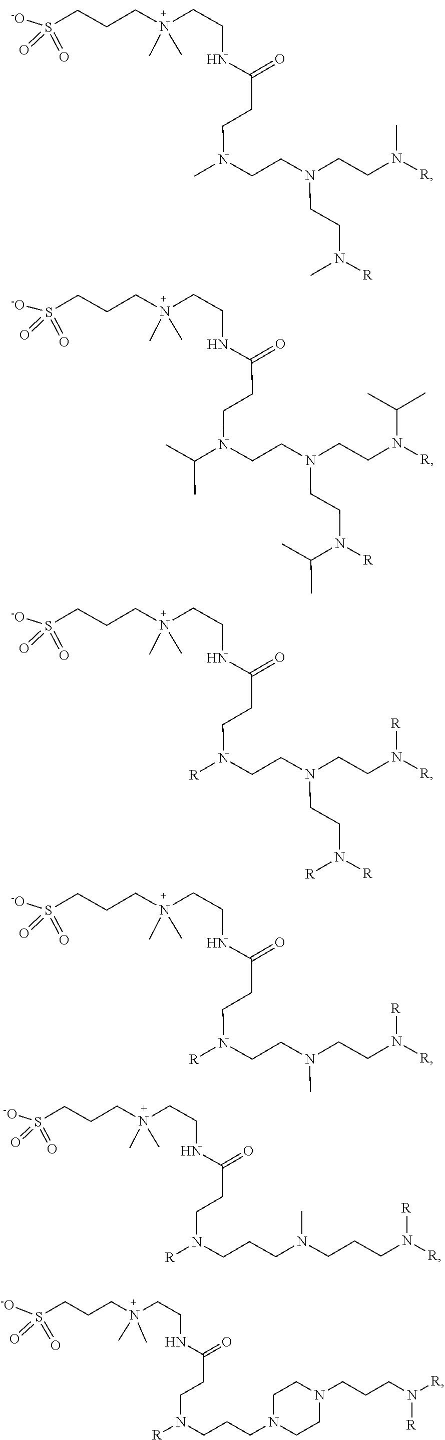
and pharmaceutically acceptable salts thereof, wherein: R is selected from the group consisting of H, —CH₂CH(OH)R₈, —CH₂CH₂C(O)OR₈, and —CH₂CH₂C(O)NHR₈, wherein: R₈ is selected from the group consisting of octyl, decyl, dodecyl, tetradecyl, hexadecyl, and octadecyl.

81. The composition of claim 80, the zwitterionic lipid has a structural formula: 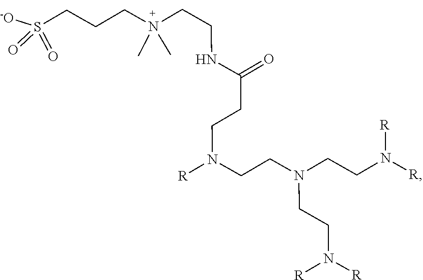
or a pharmaceutically acceptable salt thereof.

82. The composition of claim 80, the zwitterionic lipid is a compound selected from the group consisting of: 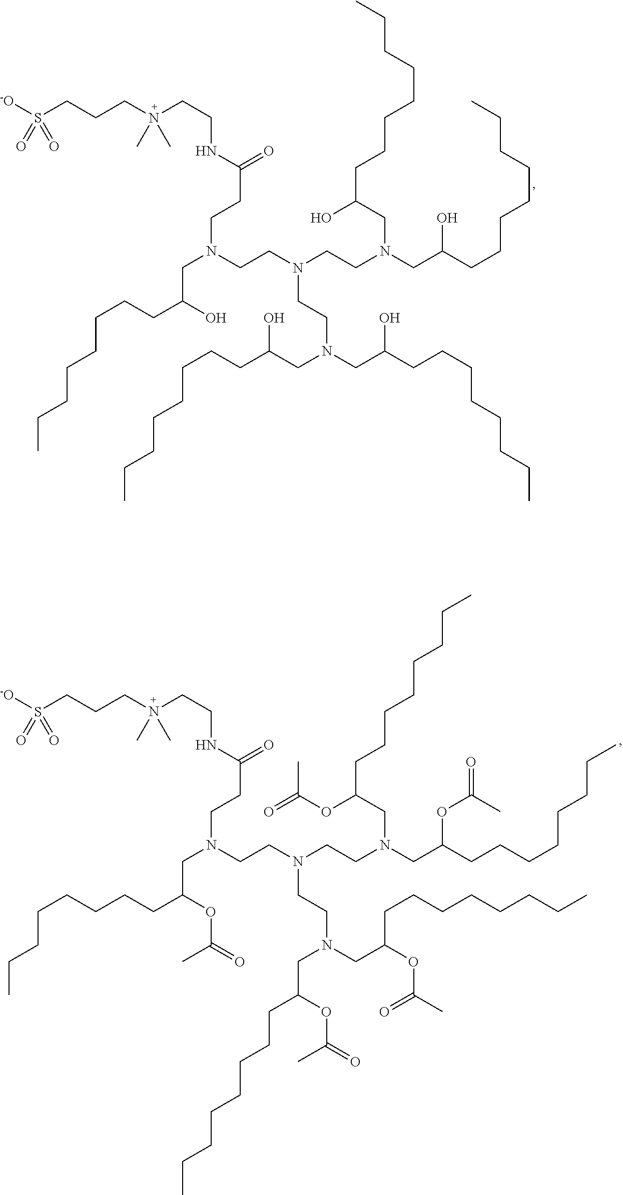 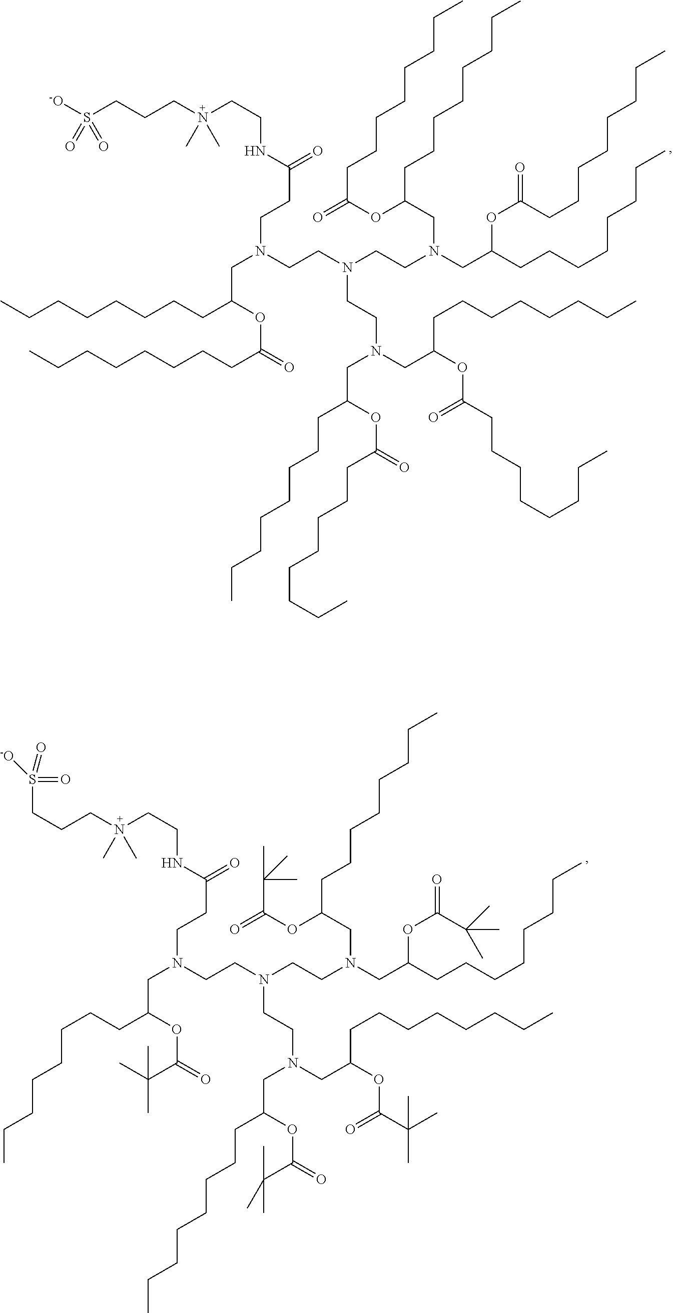 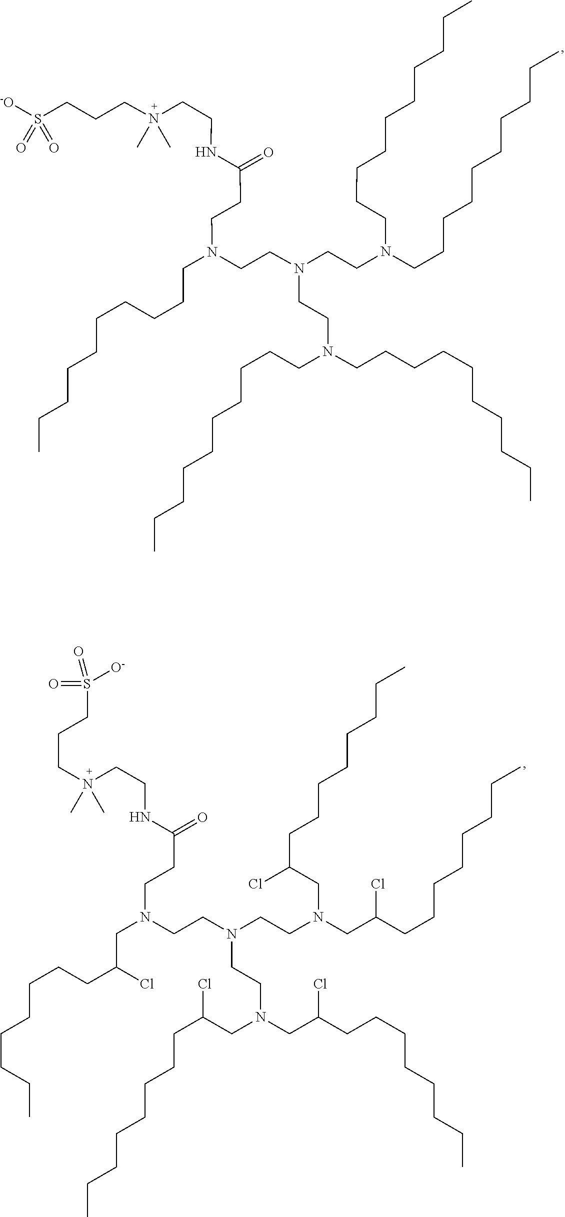 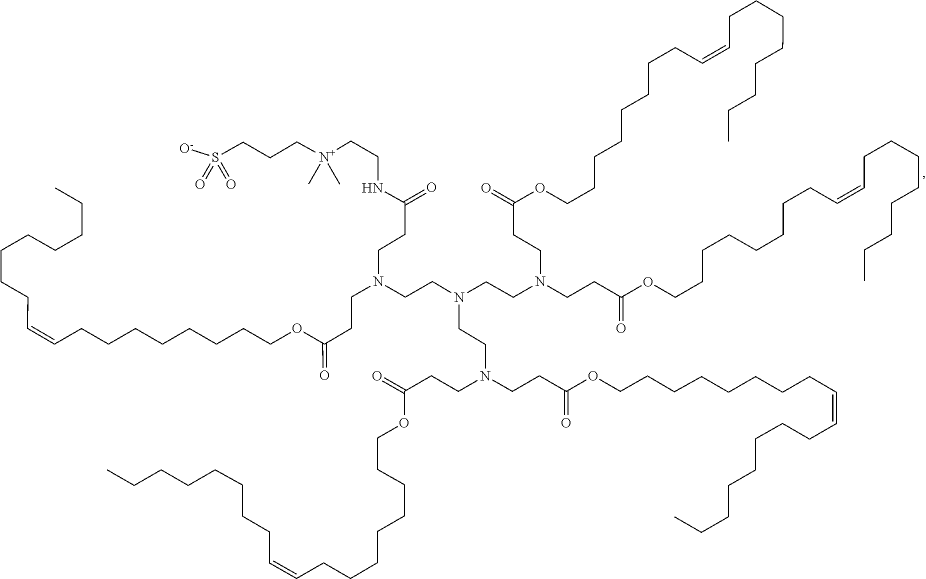 [SMILES: CCCCCCCC/C=C\CCCCCCCCOC(=O)CCN(CCC(=O)NCC[N+](C)(C)CCCS(=O)(=O)[O-])CCN(CCN(CCC(=O)OCCCCCCCC/C=C\CCCCCCCC)CCC(=O)OCCCCCCCC/C=C\CCCCCCCC)CCN(CCC(=O)OCCCCCCCC/C=C\CCCCCCCC)CCC(=O)OCCCCCCCC/C=C\CCCCCCCC]
and pharmaceutically acceptable salts thereof.

83. A method of treating a subject having or suspected of having a cystic fibrosis transmembrane conductance regulator (CFTR)-associated condition, the method comprising administering to said subject the composition of claim 66.

84. The method of claim 83, wherein said CFTR-associated condition is cystic fibrosis, hereditary emphysema, or chronic obstructive pulmonary disease (COPD).

85. The method of claim 83, wherein said administering said composition comprises a nebulization.
